(19) United States
(12) Patent Application Publication
(10) Pub. No.: US 2002/0007222 A1
(43) Pub. Date: Jan. 17, 2002

(54) Method and apparatus for supporting a body organ

(76) Inventor: Ashvin Desai, San Jose, CA (US)

Correspondence Address: PILLSBURY WINTHROP LLP, 2550 HANOVER STREET, PALO ALTO, CA, 94304, US

(21) Appl. No.: 09/910,474

(22) Filed: Jul. 20, 2001

Related U.S. Application Data
(63) Continuation-in-part of application No. 09/547,708, filed on Apr. 11, 2000.

(51) Int. Cl. A61F 2/04

(52) U.S. Cl. 623/23.65; 623/23.7; 623/902; 623/1.11

(57) ABSTRACT

A structural apparatus positioned exterior of a body organ for providing support. In one configuration, the support is in the form of a strap that has a first length with holes in which a second length with saw tooth edges is inserted to form a band to surround and give support to a body organ. In a second configuration, a sling has a central portion with saw tooth edge lengths extending in opposite directions. The central portion provides a support surface for an organ, and each saw tooth extension is embedded in part in muscle tissue, forming a supportive sling for the organ. The structural apparatus is basically constructed from polymer material that can be either bioabsorbable or non-absorbable, and alternatively can be coated with one or more layers of material including treatment substances, and additional structural additions such as cushioning, balloon material and other surface configuration.

This application is a continuation-in-part of U. S. Pat. application Ser. No. 09/547,708, filed Apr. 11, 2000, the disclosure of which is incorporated herein by reference.

### BACKGROUND OF THE INVENTION

1. Field of the Invention

The present invention relates generally to devices for supporting or lifting a prolapsed body organ, and more particularly to internal stent apparatus, and external support devices including a self locking band and a sling for maintaining the shape of an organ.

2. Description of the Prior Art

Various devices known as stents have been proposed, developed and used for placement R2 in a human body to maintain a lumen opening. Typical applications include treating occlusions of blood vessels, and urethra blockages due to benign prostate hyperplasia. Problems that 14 generally need attention in the design and use of stents include methods of insertion and removal, and prevention of stent migration. Most stents in the marketplace are constructed of a metallic coil of nitinol alloy or stainless steel. In U.S. Pat. No. 5,830,179 a stent is constructed as a coil of nitinol alloy. Nitinol is a member of a class of materials known to have “shape memory.” In practice, the wire is heated to a high temperature, wound on a mandrel or otherwise placed in a set position and cooled. The material stresses result in a “spring” tension built into the material to return to the set position as long as the material is above a certain temperature known as an Austenite state. In order to insert the stent in a body lumen, it is cooled, causing it to enter what is known as a Martensite state in which it is very malleable and can be wound on a small diameter mandrel. Once in position in the body lumen, the stent is heated, resulting in its entering back into the Austenite state, wherein the spring tension is restored, urging it back toward the set position. An alternate design uses outwardly flanged ends to provide increased resistance with the lumen wall.

### SUMMARY

It is therefore an object of the present invention to provide an improved stent that can be readily removed.

It is a further object of the present invention to provide a stent that effectively resists migration after installation.

It is another object of the present invention to provide a stent that has a coating for delivery of a treatment substance.

It is a further object of the present invention to provide a structure for surrounding an organ to provide organ support.

It is a still further object of the present invention to provide a sling for supporting a body organ.

Briefly, a preferred embodiment of the present invention includes a secure stent for maintaining a lumenal opening constructed preferably as a tubular structure of NiTi material or bioabsorbable polymer. The circumference of the tube is preferably in the shape of a polygon in contrast to the circular or oval shape of a body lumen into which the stent is to be placed. The polygon shape and ribs provide interference with the lumen wall and resist stent migration. The diameter of the stent tube is configured with each end enlarged providing flanges for interference with a lumen wall. The central portion of the stent is bulged out to an increased diameter to provide an enhanced lumen wall resistance to avoid migration. In addition, the locking feature of a ribbed structure prevents the stent from collapsing, and thereby maintains the lumen opening. The stent is preferably constructed from polymers, including bioabsorbable polymers, and/or super elastic materials. The bioabsorbable polymer construction aids removal by causing a reduction in the tube diameter as material is absorbed by body material. Attachment for removal of the stent can then be accomplished by simply grasping the proximal end of the stent. Alternatively, a stent constructed entirely of bioabsorbable material will eventually be entirely absorbed, avoiding the need for removal. Alternatively, the stent can be constructed of NiTi or other shape memory material and set in the desired shape at a high temperature. Installation is accomplished by cooling the stent to the malleable Martensite state and winding it on a small diameter mandrel of an insertion/removal tool. The compacted stent is then placed in a probe and inserted in a body lumen, whereupon it is heated to an Austenite state where it regains its spring tension, forcing it back toward the set shape. Removal is accomplished by cooling the stent to the malleable Martensite state and pulling it out. If the selected material is bioabsorbable, the stent generally does not have to be removed. Another embodiment of the present invention includes a structural apparatus positioned exterior of an organ for providing support. In one configuration the support is in the form of a band that completely surrounds the organ. In a still further embodiment a band in the form of a sling is provided for supporting a body organ with each of the two band ends anchored in muscle tissue.

### IN THE DRAWING

FIG. 1a contains side and end views of a preferred embodiment of the stent of the present invention;

FIG. 1b shows an alternate embodiment with a circular end view;

FIG. 2a shows a stent with a hexagonal cross section of constant area;

FIG. 2b shows a hexagonal stent with a concave central section;

FIG. 2c shows a stent with a hexagonal cross section and convexibulbous central section;

FIG. 3 shows a stent formed from perforated, thin flat material;

FIG. 4a is a view of flat, stepped material for forming a stent;

FIG. 4b shows the stepped material formed in an expanded spiral;

FIG. 4c shows the stepped material in a tight, compact form;

FIG. 5a is a perspective view of an expanded stent constructed with narrow, flat protrusions;

FIG. 5b shows the stent of FIG. 5a wound in a compact form;

FIG. 6a shows a stent similar to FIG. 5a with a corrugated elongated protrusion;

FIG. 6b shows the stent of FIG. 6a wound in a compact form;

FIG. 7a shows sharply and evenly corrugated sheet material;

FIG. 7b shows a stent wound from the corrugated material of FIG. 7a;

FIG. 7c shows a stent having an alternate wound form, and constructed from the material of FIG. 7a;

FIG. 7d shows a stent wound from material with alternating abrupt and tapered lengths;

FIG. 7e illustrates a stent wound from a corrugated material with abrupt points separated by curved sections;

FIG. 7f shows a stent wound from continuously curved corrugated material;

FIG. 7g illustrates holes in stent material;

FIG. 8 illustrates the use of a turn block to expand and contract the cross section of a stent;

FIG. 9 shows a scissor-jack for expanding and contracting a stent;

FIG. 10 illustrates the use of a biodegradable coating over a stent base;

FIG. 11 is a list of bio-absorbable/biodegradable materials;

FIG. 12 is a list of anti-microbial coating materials;

FIG. 13 lists coating materials that can be used as lubricants;

FIG. 14 is a list of drugs/pharmaceuticals, etc. for inclusion in a stent coating;

FIG. 15 shows a stent base of smaller diameter with perforations through which a material can be ejected to secure the stent base to a body lumen wall;

FIG. 16 illustrates a stent in the form of a balloon;

FIG. 17a illustrates an endoscopic instrument for inserting a stent;

FIG. 17b is an expanded view of a stent and ejection device in reference to FIG. 17a;

FIG. 18 shows a polycatheter and balloon device for inserting a stent;

FIG. 19 illustrates a simple stent installation tool;

FIG. 20a shows a collapsed organ;

FIG. 20b illustrates the use of a strap to support the collapsed organ of FIG. 20a;

FIG. 20c is a planar view of the strap shown in FIG. 20b in its linear, relaxed state;

FIG. 21a is a planar view of a sling for supporting an organ;

FIG. 21b illustrates the use of the sling of FIG. 21a to support a body organ;

FIG. 22 shows a tool for use in installing a support member;

FIG. 23 illustrates the use of the tool of FIG. 22 for installation of a support member;

FIG. 24 shows a strap installed around an organ in a body cavity;

FIG. 25 is a cross sectional view of a support member for illustration of a bubble layer applied over a polymer layer;

FIG. 26 is a cross sectional view illustrating the application of various layers over a polymer layer;

FIG. 27 illustrates a strap having multiple saw tooth lengths;

FIG. 28a shows a sling constructed as a balloon for providing an alternative method of adjustment;

FIG. 28b is a cross section of the balloon sling of FIG. 28a in a relatively collapsed state;

FIG. 28c is a cross sectional view of a balloon sling, illustrating application of an Gil additional layer of material over the balloon structure;

FIG. 29a illustrates a sling with adjustable length extensions;

FIG. 29b shows the shortened adjustable length of the adjustable sling;

FIG. 29c illustrates securing the shortened length; and

FIG. 30c illustrates use of an injectable support material.

### DETAILED DESCRIPTION OF THE PREFERRED EMBODIMENT

A preferred embodiment of the present invention is illustrated in FIG. 1a wherein a tubular stent 10 formed from coiled wire 11 is shown in a longitudinal view 12 and an end view 14. The present invention includes longitudinal variations in the stent cross section, the longitudinal direction defined by axis 16. The stent 10 has a flared proximal end 18 and a flared distal end 20. The middle portion 12 is bulged out. The combination of these variations in the cross section, ie. variations in the distance of the tube wall from axis 16 as a function of distance along the axis 16, including flared ends 18, 20 and the bulged midportion 22 results in a stent with an increased strength to retain a lumen wall, and an increase in resistance to stent migration/movement in a body lumen. The bulged central/midportion 22 is important in that it provides greater strength in resisting lumen wall pressure than a straight tube section would provide. The stent wall can be constructed from any of various biologically compatible materials, such as NiTi, stainless steel, and various biodegradable polymers. The benefit of the construction is that pressure on the rim 24 of the flares 18, 20 is transferred in part to bulbous midsection 22, giving it greater strength. The end view 14 illustrates another feature of the present invention, showing the outline of the rim 24 of the distal end 20 of the flare. This hexagonal shape continues for the entire length of the stent 10, varying in area from a maximum at the ends 18, 20 and in the middle 22 to a minimum contour 26 between the midsection and flared ends. The hexagonal shape is a preferred embodiment, but other irregular shapes are also included in the spirit of the present invention. The novel purpose of an irregular outline for a stent is to provide increased frictional contact with a typically round or oval shaped lumen wall. The pressure of the irregular shaped stent against the lumen wall causes the wall to expand and partially conform to the stent outline. The irregular shaped stent contour provides areas (for example at 28) of increased pressure, resulting in more resistance with the body lumen wall than would occur if the stent outline were round or oval, such as illustrated in the alternate embodiment of FIG. 1b.

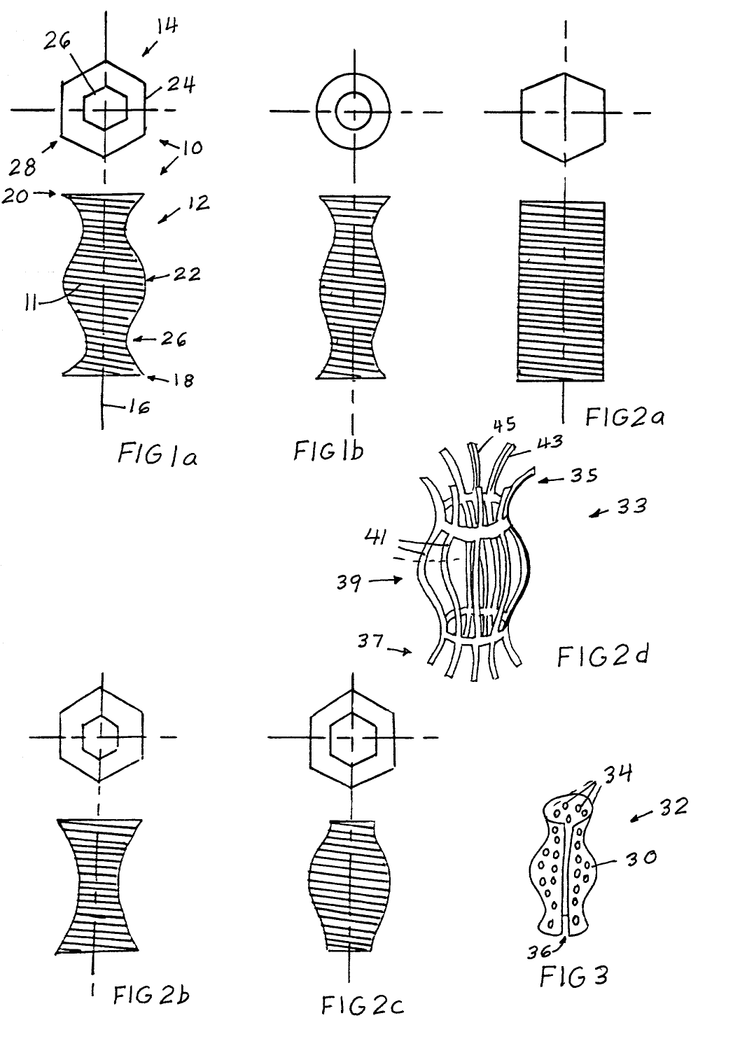

FIG. 2a shows an embodiment utilizing only the irregular cross-section feature, without the variation in cross-section over the length of the stent. FIGS. 2b illustrates the use of the irregular cross-section combined with flared ends. FIG. 2c shows a stent with only a bulged middle.

FIG. 3 shows the use of a thin sheet material 30 to form a stent 32. The perforations 34 are optional. The slot 34 allows the stent 32 to be readily collapsed for insertion and removal.

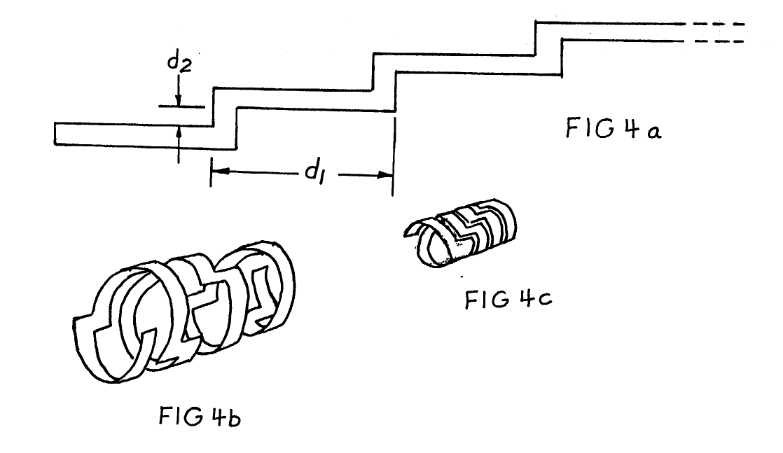

FIGS. 4a-4c illustrate a stent construction using a flat ribbon type of material that is cut in steps as shown in FIG. 4a. The steps are therefore formed in the plane of the flat, sheet/ribbon material as distinguished from steps or corrugations that will be shown in subsequent figures of the drawing. When the material of FIG. 4a is wound on a mandrel, it has an expanded form as shown in FIG. 4b. It can be heated and set in the expanded configuration of FIG. 4b, and then compressed by further winding to a smaller configuration such as FIG. 4c. The step lengths “d₁” determine the minimum circumference of the tightly wound stent as shown in FIG. 4c. Each “turn” of the stent is spaced from the next by the distance d₂ which can be any value desired.

FIGS. 5a and 5b illustrate another alternate stent 38 embodiment. Constructed from flat material of width “w”, it is bent, forming a plurality of short protrusions 40 of lengths h₁ and a single elongated protrusion 42 of length L. The protrusions 40 and 42 are joined with a radius R, if allowing an open lumen 44 through the full length of the stent which is the width “w” of the flat material. The stent 38 is placed in a cylindrical shape as shown in FIG. 5b by winding the elongated protrusion 42 around the axis 46 of the stent lumen 44, in the process folding/bending over the short protrusions 40 resulting in a compressed stent 38 of small diameter D for insertion into a body lumen. FIGS. 6a and 6b illustrate an alternate embodiment 48 of the same general type as shown in FIGS. 5a and 5b. he elongated protrusion 50 has a corrugated side 52 that is included to increase contact resistance with a body lumen wall to reduce stent migration. FIG. 5b shows the compressed, wound state of the stent, clearly showing the corrugated side 52 facing outward. This figure also clearly illustrates bent shorter protrusions and a stent lumen 56 that are features in common with the stent 38 of FIGS. 5a and 5b.

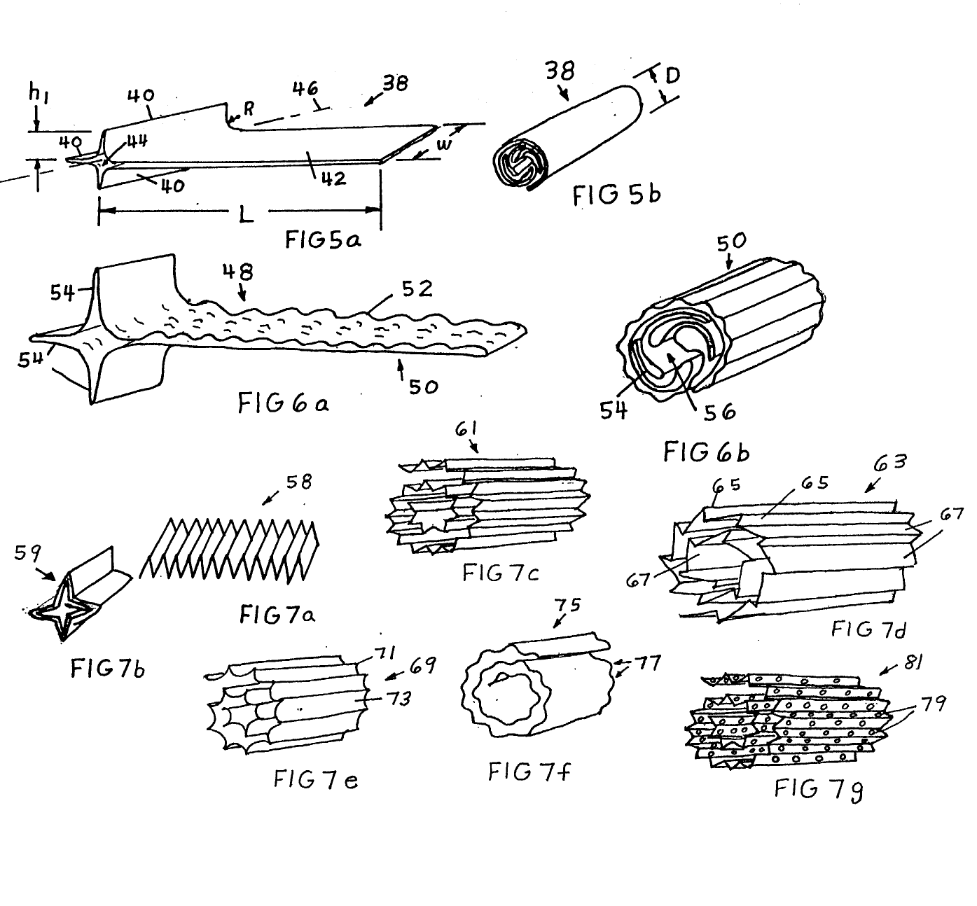

A further alternate stent embodiment 58 is shown in its wound compressed state in FIG. 7a. It is formed from a corrugated material 59 as shown in FIG. 7b. Additional alternate stent embodiments constructed from corrugated sheet material are shown in FIGS. 7a-7g. Shown in FIG. 7a is an evenly bent material 58 which can be wound to form stents 59 and 61 as shown in FIGS. 7b and 7c.

FIG. 7d shows a similar stent 63, differing from stent 61 in that the sheet material is bent so as to provide abrupt ridges 65, which interfere with each other to resist winding once the stent 63 is expanded, providing a self-locking feature.

In fact, all of the stents of FIGS. 7b-7f provide a degree of resistance to compression/rewinding due to the resistance provided by interfering corrugations. Stent 75 of FIG. 7f provides the least resistance, having smoothly formed corrugations. Expansion is encouraged in the stent 61 design of FIG. 7d by the more gently sloping ramps 67.

The stent 69 of FIG. 7e uses ridges 71 separated by curved portions 73. In FIG. 7f the stent 75 is constructed of continuously curved corrugations 77. Any of the stents constructed of sheet material can also have holes, such as holes 79 in stent 81 of FIG. 7g.

The stents of FIGS. 1-7 are preferably constructed of a shape memory material and heat set in an expanded configuration in the Austenite state. In order to insert the stent in a body lumen, it is cooled to the Martensite state wherein the material becomes malleable, lacking resiliency. In this state, the material can be reformed to a compact state. In this compact state, it can be inserted into a body lumen. The stent is preferably placed on a mandrel that is part of an insertion tool prior to cooling and compacting.

A preferred shape memory material is nitinol (NiTi), but the present invention includes the use of other shape memory materials that will be apparent to those skilled in the art. In addition, the stent material can be a biodegradable material, such as a biodegradable polymer. The stents can also be made from a combination of biodegradable and non-degradable materials. For example, in FIG. 7f, the outer layer can be constructed from a biodegradable material, and the inner layer can be constructed of a non-biodegradable material. In this case, when the outer layer is absorbed, the inner layer can be removed.

The stents can also be constructed from nitinol or other super elastic material, processed/heat-treated to what is known as a “super elastic” state. In this state the material retains its resiliency at lower temperatures, and can be used for a permanent stent installation. Removal would require use of a tool to cut or compress the stent.

The stents of FIGS. 1a through 2c, and FIGS. 4b, 5a, 6a and 7b are all shown in an expanded state. When they are constructed of a shape memory material and heat set in this expanded state, they can then be cooled and wound or otherwise compressed to minimize the size during insertion into body lumen. The shape of FIGS. 4c, 5b, and 6b are all examples of a Eli compressed stent in its Martensite state. After insertion in a body lumen, the temperature rises and the material returns to the Austenite state, regaining its resiliency, and causing a force against a body lumen wall in the effort to return to the original state. The stent 58 as shown in FIG. 7a can conceivably be further compressed for insertion by bending the protrusions.

As mentioned above, the stents as disclosed herein can be made out of any biocompatible material that will allow some method of insertion and removal from a body lumen. The stents of FIGS. 1-7 could be constructed of a permanently resilient material such as stainless steel, and could be collapsed with some difficulty for installation in a probe for insertion. However, removal in such a case would generally require a forceps. Constructing the stents of FIGS. 1-7 with a shape memory material as discussed above is preferred. After cooling the stent, it can be wound, folded, collapsed, etc. as required in order to be loaded into a probe lumen for transport into a body lumen. A push rod in back of the stent in the probe lumen can be used to eject the stent once the probe is in the desired location. As discussed above, the stent is then simply heated, by any of various means including body temperature or a warrn saline solution to bring the stent back to the Austenite state wherein it regains its original resiliency. Removal is accomplished by injecting a cool saline solution to bring the stent back to the malleable Martensite state, whereupon it can be readily pulled out.

The flared ends 18, 10 of FIG. 1 provide resistance with the body lumen wall, keeping the stent from moving in the lumen. The bulbous portion 22 is placed where maximum body lumen enlargement is required. The force of the body lumen on the stent portion 22 tends to cause the ends 18 and 20 to expand, which provides enhanced resistance with the lumen walls to avoid migration. The force of the ends 18 and 20 on the body lumen wall is also reflected back to provide resistance to compression of portion 22.

Other shapes for the circumference/cross-section of the stents are also included in the spirit of the present invention. For example, the polygon shape in FIG. 1 could be square, fivesided, an octagon as shown, etc., or other irregular shape to increase resistance between the stent and the body lumen wall. The present invention also includes a circular or oval circumference, as indicated in FIG. 1b.

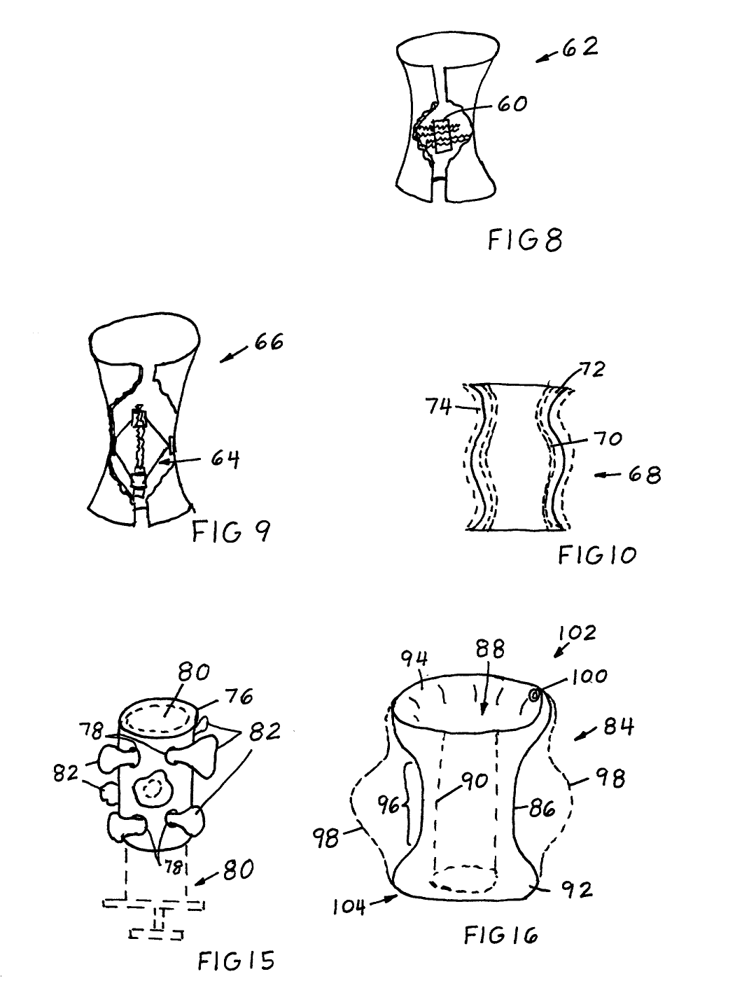

An alternate method of collapsing and expanding a stent is illustrated in FIGS. 8 and 9. An expansion and contraction apparatus can be installed inside a stent that is constructed of sheet material. FIG. 8 shows a turn block 60 that can be activated to contract the diameter of a stent 62 for insertion in a body lumen. The turn block can then be applied to expand the stent against the lumen wall. When removal is required, the reverse procedure is applied. FIG. 9 shows a similar arrangement where a scissor apparatus 64 (similar to a small car jack) is used to expand and contract the diameter of a stent 66.

FIG. 10 illustrates another embodiment of a stent 68 that is designed for temporary use. NiTi or other biologically compatible material 70 is used to form a stent base. A coating of biodegradable material 72 is placed over the base 70. The base can optionally also be made of biodegradable material. The base 70 can be of any desirable configuration that can be collapsed for insertion in a probe lumen for installation in a body lumen, including structures similar to those of FIGS. 1-7. The expanded size of the base 70 is preferably small enough to clear the size of the body lumen into which it is to be placed, if it is not biodegradable, so that when the material 72 is absorbed by the body, the base 70 can be easily removed. A second coating 74, or first coating if coating 72 is omitted, can be included. The coating 74 is generally for inclusion of some type of treatment substance, but can be for any purpose, including the purpose of providing interference with the body lumen walls. If coatings 72 and/or 74 are for determining the stent size, the selection of material 72 and thickness depend on how long the stent is to remain in the body. After the material has been sufficiently absorbed, the stent base can be removed by simply grasping it with an appropriate device through an endoscope lumen, for example. This procedure avoids the need to compress the stent for removal, although collapsible stents can also be used. If the stent base is biodegradable, it will eventually be absorbed, and may therefore not have to be removed.

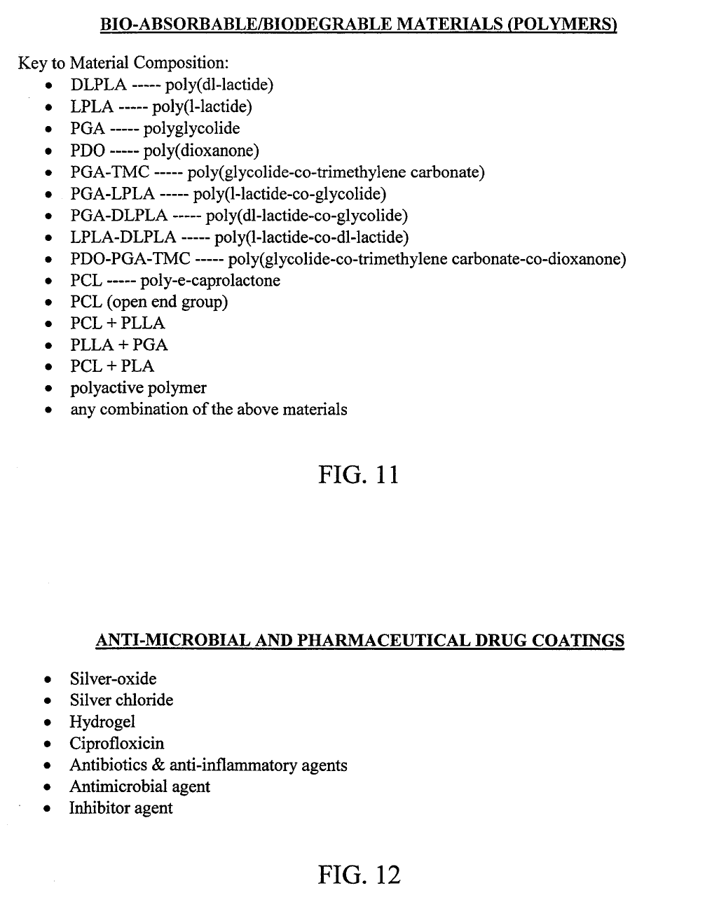

FIG. 11 lists various biodegradable materials that can be used to coat a stent, as indicated above. The stents, or stent bases, described above can be constructed from NiTi or a biodegradable polymer, or any other appropriate material known to those skilled in the art, such as stainless steel or any of various compatible polymers. As mentioned above, stents can be constructed entirely from biodegradable material. A number of these are listed in FIG. 11 and will be recognized by those skilled in the art as applicable to the construction of the stent designs disclosed herein. The benefit of using an all biodegradable material is to avoid the necessity of any removal of a stent.

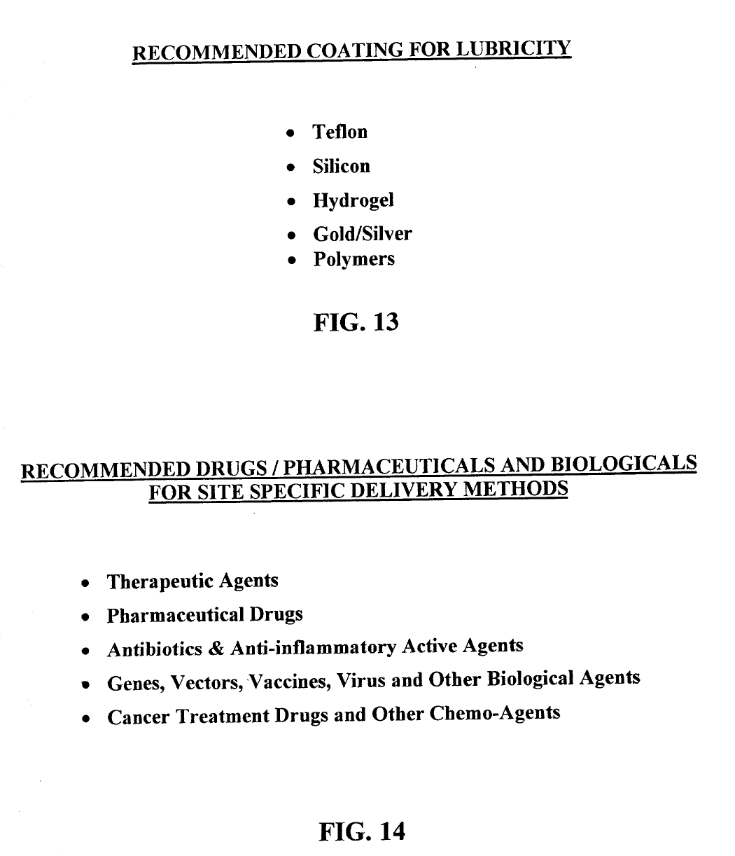

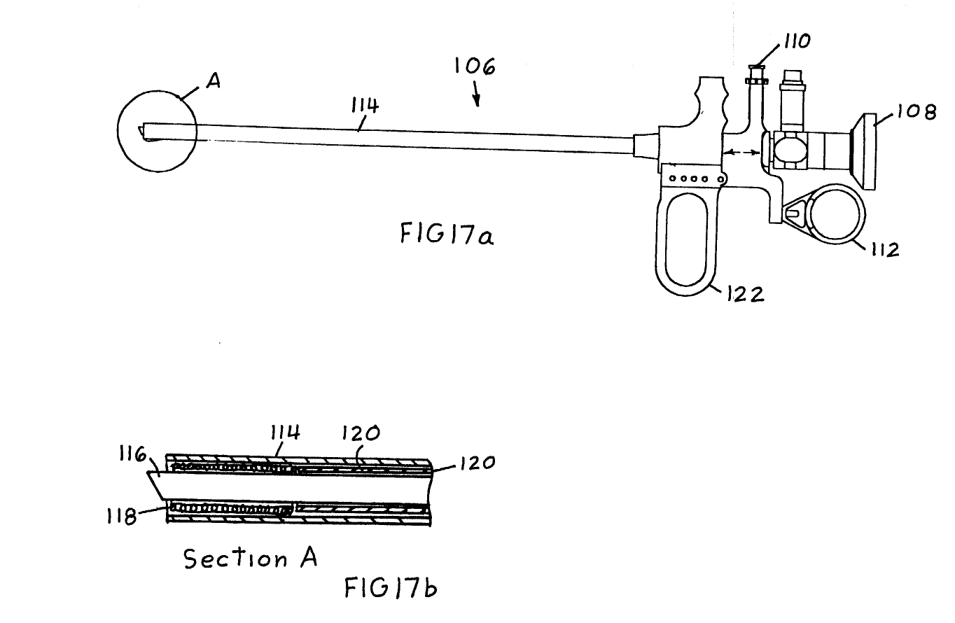

Coating materials for layer 74, as discussed above in reference to FIG. 10, include, but are not limited to those listed in FIGS. 12, 13 and 14. FIG. 12 lists anti-microbial coating materials to reduce the possibility of infection. FIG. 13 lists a selection of materials that reduce friction, i.e. for lubrication. FIG. 14 lists various drugs/pharmaceuticals, etc. as examples of potentially beneficial materials that can be applied to the stent to provide a localized treatment of body tissues.

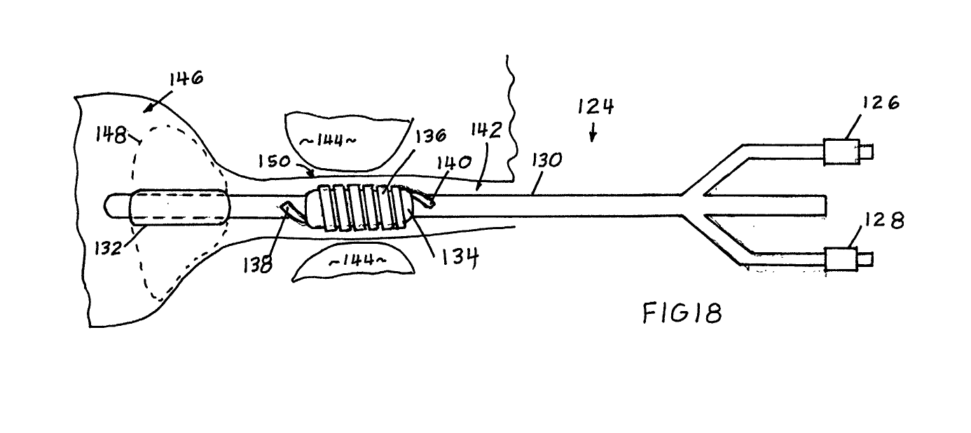

Another embodiment of the present invention is illustrated in FIG. 15 wherein a cylindrical stent base 76 includes a plurality of holes 78. The stent base 76 is inserted in place in a body lumen. An injector probe, symbolically illustrated as item 80, is then inserted inside the base 76, and a bio-compatible material 82 is injected and forced out the holes 78 and against the wall of the body lumen (not shown). The material 82 would preferably be constructed to harden i.e. set-up quickly after injection.

A still further embodiment of the present invention is illustrated in reference to FIG. 16 wherein an inflatable balloon is used as a temporary stent. The balloon 84 is shown in its uninflated state by solid lines 86. The balloon has a lumen 88 therethrough. The walls of the balloon are constructed with variations of thickness to force expansion in desired directions. The wall 90 of the lumen 88, and the walls of the flared end sections 92, 94 are thicker to avoid expansion. The wall 96 of the outside center portion is thinner to force expansion upon balloon inflation, the inflated state indicated by the dashed lines 98. A self-sealing inflation port 100 is provided on a proximal end 102. The distal end 104 is inserted first in the body lumen.

A method and apparatus for inserting a stent is illustrated in FIGS. 17a and 17b utilizing an endoscopic instrument 106. The instrument 106 has a telescope 108, an irrigation/aspiration port 110, and a slide trigger 112. Section “A” of FIG. 17a is shown enlargened in FIG. 17b. The endoscopic instrument 106 includes a probe 114, in which is inserted a first tube 116 through which a telescope probe and/or instrument can be passed. A stent 118 is assembled over the first tube 116. In order to eject the stent from the tube 116, a second tube 120 is provided encircling a the first tube 116. The second tube 120 is linked to the slide trigger 112 and pressing the trigger 112 in toward handle 122 moves the second tube 120 to eject the stent from the probe 114. With the stent in place in the body lumen, it will immediately expand if it is constructed from a permanently resilient material such as stainless steel. If it is constructed from a shape memory material such as Nitinal (NᵢNᵢ) it would have been cooled prior to insertion and compressed in the Martensite state. When it is in place in the body lumen, it expands when its temperature is raised, bringing it back into the Austenite state and regaining its resiliency. Removal of the shape memory material is accomplished by cooling the stent to bring it into the maleable Martensite state and then simply pulling it out.

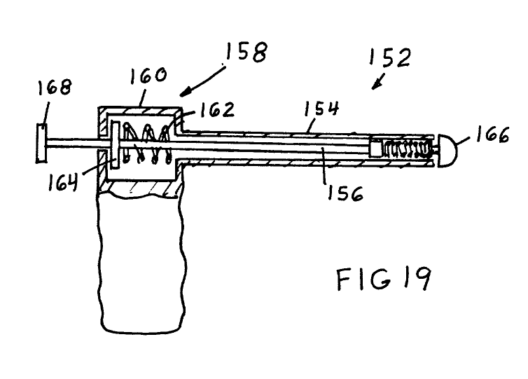

FIG. 18 illustrates another tool that can be used to insert a stsent, including a polycatheter 124 having two one way valves 126 and 128. The catheter 124 has a probe 130 upon which is mounted a first balloon 132 supplied with air by way of valve 126 and a second balloon 134 supplied with air by way of valve 128. A stent 136 is positioned around the second balloon 134. The stent 134 shown in FIG. 18 is formed from flat ribbon material for ease of illustration, but any of the stents disclosed above in FIGS. 1-4, as well as others that will be apparent to those skilled in the art can be installed. The ends 138, 140 of the stent material are releasably attached to the probe 130. The method of attachment can be through use of an adhesive, or by way of other fragile connection.

Insertion of the stent 136 is illustrated in FIG. 18, with the catheter inserted in a urinary tract 142 and the stent 136 positioned adjacent the prostate 144. The first balloon 132 is positioned just inside the bladder 146 at this point in the procedure. Air is applied to the first balloon 132 through one way valve 126, expanding it as shown by dashed lines 148 to contact the bladder wall, securing the catheter 124 in place. The second balloon is then inflated through valve 128. As the balloon 134 expands, tension is placed on the attachment of the ends 138 and 140 until they break, freeing the stent 136 to expand against the wall 150 of the urinary tract 142. If the stent is stainless steel or other permanently resilient material, it will immediately expand upon breaking the attachment ends 138 and 140. If the stent is a shape memory material, it will expand after first being raised in temperature to the Austenite state, which may occur from body temperature or by injection of a heated solution into the urinary tract.

FIG. 19 is a simplified sketch for illustrating some of the features of a stent insertion tool. The tool 152 includes a body probe 154for insertion in a body lumen, and an installation probe 156. An apparatus 158 includes a housing 160, a spring 162, and plate 164 attached to the probe 156, all configured to apply a spring force to retain the probe 156 inside probe 154 during traversal of a body lumen. With the probe head 166 in place, an operator pushes on the button 168, impelling the stent as explained above. Upon releasing the button, the spring 162 retracts the probe 156.

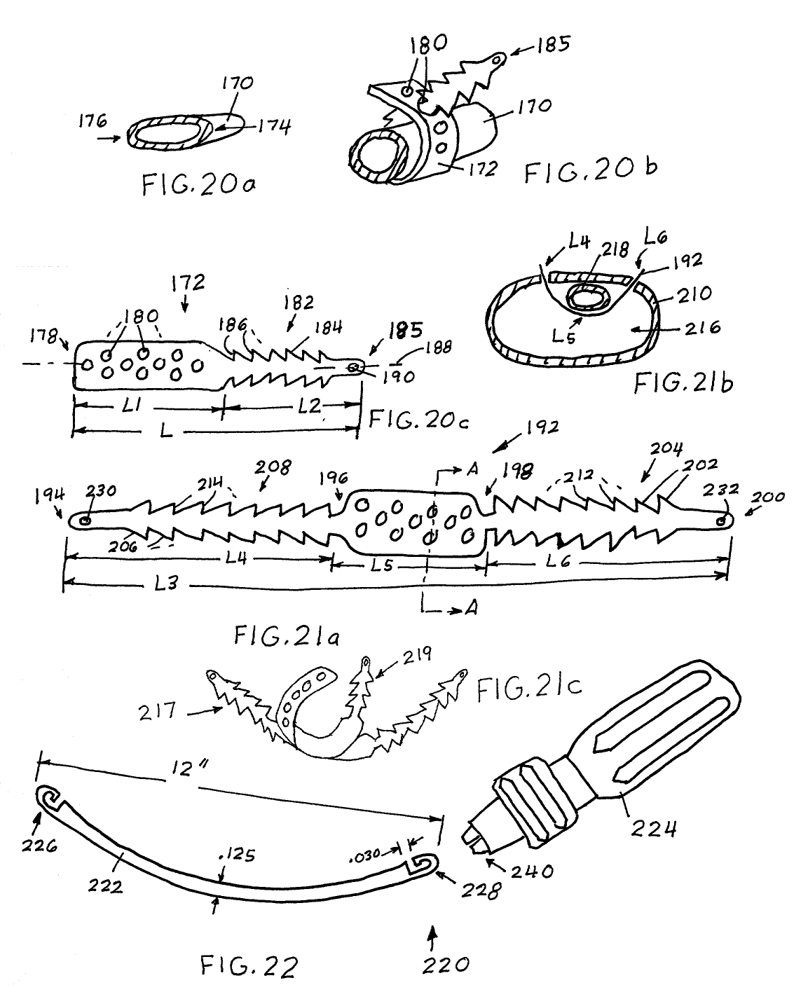

A further embodiment of the present invention includes a method and apparatus positioned on the exterior of a body organ for adding structural support or lifting/suspending the organ. According to one aspect of the method of the present invention, an organ that is in a collapsed state can be supported or suspended with an external strap to hold the organ in its normal shape and position. FIG. 20a, for example, shows a cross section of an organ 170 that is partially collapsed. FIG. 20b shows the organ 170 with a strap 172 that serves to push the sides 174 and 176 (FIG. 20a) inward to cause the organ 170 to be restored to its more normal anatomical position and configuration as shown in FIG. 20b. A specific example of the use of the strap is to correct sphincter deficiency in either a male or female. The strap 172 is more clearly shown in its uncoiled state in FIG. 20c. The strap 172 has a length “L” and from a first end 178 has a first length “L₁” perforated with a plurality of holes 180 or other cut outs of any shape, followed by a second portion “L₂” with a plurality of protruding spikes, which can be spherical, triangular, pyramid shape, etc., or various other configurations as will be apparent to those skilled in the art, but which are shown preferably as saw tooth shaped protrusions 182 with tapered edges 184, each facing forward to an opposite second end 185 of the strap 172, and having a more laterally oriented edge 186 directed outward from the strap centerline 188. A guide hole 190 is formed through the strap 172 near the second end 185.

Referring again to FIG. 20b, the strap 172 is bent around the organ 170 and the second end 185 is fed through one of the plurality of holes 180 until the strap is in the shape of a constricted loop sufficient to add the required support/radial force to the organ 170. The strap in the area of the saw tooth protrusions 182 is wider than the diameter of the holes 180, but is flexible enough to bend so as to allow the protrusions to be forced through a selected hole 180. Once the required length of portion L₂ is in place as shown in FIG. 20b, the laterally oriented edges 186 resist backward passage of the length L₂ through the hole 180, making it self locking.

The strap 172 can be used to prevent prolapse of any type of body organ 170 in need of being reinforced, such as a urethra, uterus, bladder, colon, vagina, rectum or any body organ, etc. The support apparatus of the present invention, including the strap 172 and other straps and slings as described in reference to the following Figures of the drawing, are preferably constructed from a polymer of copolymer, either bio-absorbable or non-absorbable, or any combination of these and other materials as indicated in the following description and claims. For example, the apparatus can be constructed from a non-absorbable polymer or a super elastic material, coated with a bio-absorbable polymer. The support apparatus can be made of various configurations, as in the form of a flat ribbon, which can be constructed by material interleaving such as woven or knitted fibers, or it can be constructed from composites or of a molded/extruded construction to achieve the desired tensile strength, and of dimensions selected to provide sufficient time before complete absorption if constructed from bio-absorbable material. As a still further embodiment, the construction can include a bio-absorbable polymer with a color additive, and/or a bio-absorbable polymer coated with a layer including a medication such as an antibacterial/antimicrobial material or a pharmaceutical drug or other chemical agent or gel for delivery to the tissues.

Another embodiment of a support structure of the present invention is in the form a suspension sling 192 as illustrated in FIG. 21a. The sling 192 has a length L₃ with a first portion L₄ extending from one end 194 of the sling 192 to a first end 196 of a central portion of length L₅. A third portion of length L₆ extends from a second end 198 of the control portion to a second end 200 of the sling 192. The lengths L₆ and L₄ are configured in a similar way to the length L₂ of the strap 172 of FIG. 20c. The spikes, or as shown saw toothed protrusions 204 of length L₆ and the saw toothed protrusions 208 of length L₄ are for anchoring L₄ and L₆ in muscle tissue to form a self anchoring sling with the central portion L₅ giving support to an organ. The tapered edges 202 and 206 point away from the central portion L₅ The abrupt, laterally directed edges 212 and 214 resist backward motion of the length L₆ once set in a muscle tissue. This is illustrated in FIG. 21b wherein the sling 192 has lengths L₄ and L₆ passing through muscle tissue 210. The lateral edges such as 212 and 214 (FIG. 21a) resist motion of the sling portions L₄ and L₆ back into the body cavity 216. The central portion L₅ is shown to give support to a body organ 218. The material of which sling 192 is constructed is from the same selection as that described above for the strap 172. In an alternate embodiment lengths L₄ and L₆ can be anchored to bone or surrounding muscle tissue using any of various means that will be apparent to those skilled in the art, such as with staples, sutures, pins, etc. The sling concept of the present invention is not limited to the single length as shown in FIGS. 21a and 28a. The sling can also include a loop around the organ to provide the support described in reference to FIG. 24, and/or both a sling as shown in FIGS. 21a and 28a and a band as shown in FIG. 24 can be used. A combination sling 217 with integrated band 219 is shown in FIG. 21c.

FIG. 22 illustrates a tool 220 for use in installing a strap or sling. The tool 220 has a probe 222 portion and a handle 224. Each end 226 and 228 of the probe 222 is configured in a hooked shape for engaging with the guide holes of the support structures, such as hole 190 of strap 172 and holes 230 and 232 of sling 192. The ends of the probe 222 can be of various shapes for grabbing the support structure. For example, the probe end can be an eye hook, J-hook, L-hook, S-hook etc., or it can be in a triangle or ball shape, etc. Other methods of threading the structures into a body will be apparent to those skilled in the art, and these are also to be included in the spirit of the present invention.

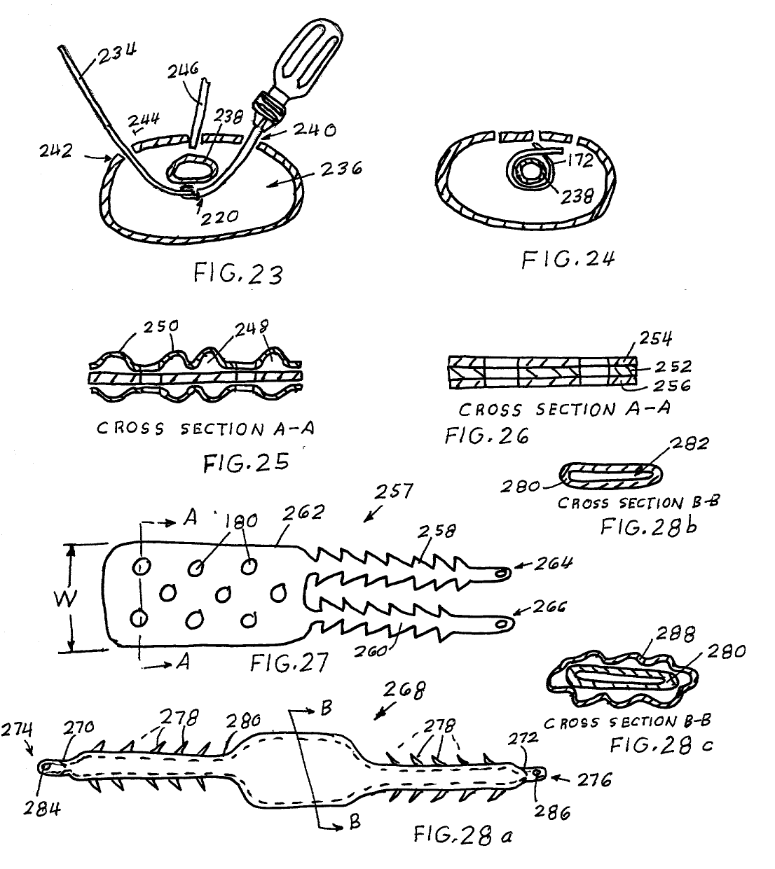

FIG. 23 illustrates a use of the tool 220 in the installation of a support structure 234 in a body cavity 236 for supporting an organ 238 such as urethra. The modular handle 224 is designed with a pin vise grip 240 or other locking mechanism (FIG. 22) so that it can grip either end 226 or 228 of the probe 222. The probe 222 as shown in FIG. 23 has been inserted through a first perforation 240, around the organ 238 and out a second perforation 242. The suspension structure 234 is then attached at one end 244, after which the tool 220 can be used to pull the structure 234 in or out of the cavity 236. If the structure 234 is the suspension sling 192, the end 244 is the same as end 200 of the sling 192, which is pulled all the way through and out perforation 240, with the end 200 protruding from perforation 240, and the first end 194 (FIG. 21a) left outside perforation 242, the result being as illustrated in FIG. 21b. FIG. 23 also shows an endoscope probe 246 inserted into the body cavity for viewing any desired part of the procedure. The endoscope can also be inserted into the collapsed organ to make sure that the organ is not punctured by the insertion tool 220. If the structure 234 is a strap such as strap 172 of FIG. 21c, the tool 220 can be used for grabbing the end 185 (FIG. 21c) and pulling the strap around the organ. The tool and strap can be viewed inside the body during the procedure with an endoscope. Another tool 220 can be attached to the other end 178 of the strap and follow the strap around the organ. Once inside, the operator can view through the endoscope to use the two tools 220 to thread/push the end 185 around the organ 238 and through a selected hole 180 to surround the organ as shown in FIG. 24.

As a further alternate embodiment, the support structures of FIGS. 20c and 21a can be constructed with a surface texture, which can be ribbed, embossed or shaped in any other way known to those skilled in the art to enhance tissue adherence and/or growth. For example, the material can be constructed to include bubbles containing gel or other media. This is illustrated in FIG. 25 which is a cross section view A-A as indicated in reference to FIGS. 20c and 21a. The structure of FIG. 25 is shown with three layers including bubbles 248. FIG. 25 is also used to illustrate a general surface texture which, for example could be a single layer with an external surface texture, or as a multiple layered structure as shown. The bubbles 248 of the multiple layered structure shown, can be filled with a gas such as air, or a gel or fluid, etc. for cushioning, or with a treatment substance and/or drugs that can be time released if the bubble casing material 250 is bio-absorbable/biodegradable. FIG. 26 is also a cross section A-A in reference to FIGS. 20c and 21a. FIG. 26 illustrates a layered construction without bubbles. A central layer 252 can be constructed from any bio-compatible polymer, and can be either biodegradable or non-biodegradable or any other material. Layers of material or composite can be added to either or both sides of the central layer 252. Layers 254 and/or 256 can be constructed from any bio-compatible material. A preferred embodiment includes layers 254 and/or 256 consisting of bio-absorbable material including treatment substances or chemical agents. Although FIG. 26 shows three layers, the present invention includes any number of layers constructed from any bio-compatible material. FIG. 26 shows a cross section A-A through the perforation of the strap and sling, but the multiple layer and material composition also apply for use in portions that are saw toothed as an alternate embodiment.

The list of bio-absorbable/biodegradable materials of FIG. 11, and the list of lubricating coatings of FIG. 13, and the lists of treatment substances of FIGS. 12 and 14 all apply as selectable materials for construction of the structures of FIGS. 20-26 above as well as to those shown in the following figures of the drawing.

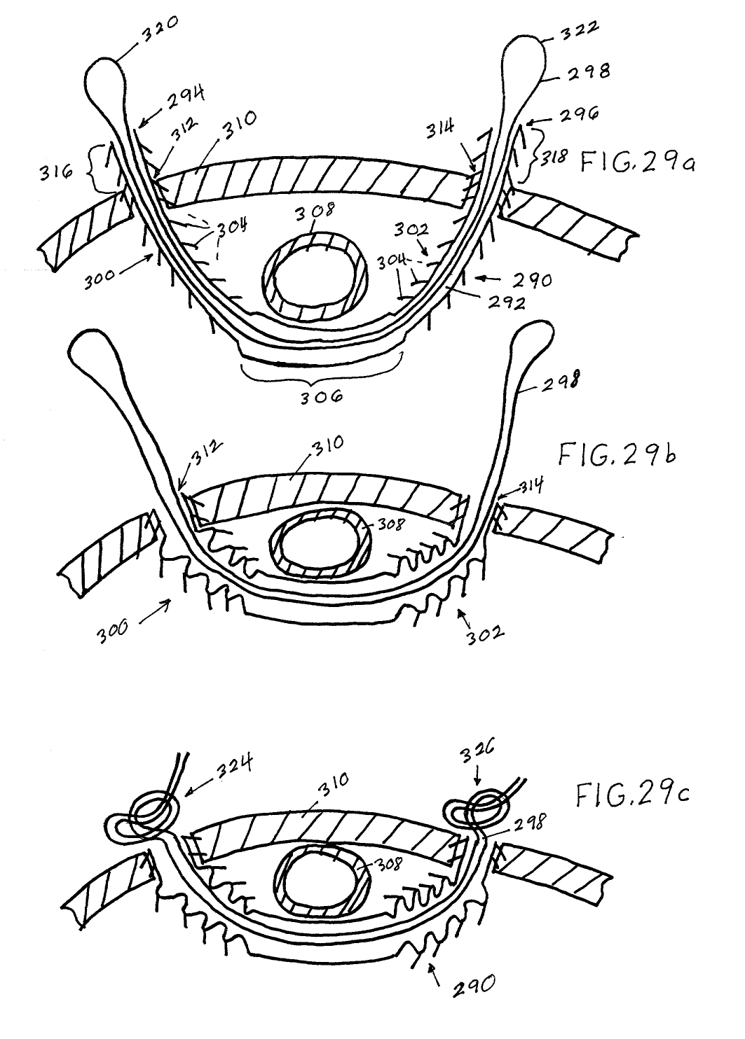

FIG. 27 shows an alternate embodiment of a strap 257 having two lengths 258 and 260 with spikes that can be saw toothed as shown or other shaped configuration. The perforated section 262 is similar to portions L₁and L₅ of FIGS. 20c and 21a, and the above comments regarding the cross section A-A of FIGS. 25 and 26 also apply to the apparatus of FIG. 27. In the case of FIG. 27, each end 264 and 266 is threaded through a selected one of holes 180 in the operation of securing the strap 257 around a body organ. The two straps 264 and 266 allow a wider width W of support. Although FIG. 27 shows two saw toothed/spiked lengths 258 and 260, the present invention includes any number of spiked lengths as may be desired for the intended purpose of providing adequate support. An additional benefit of a plurality of spiked lengths is the ability to provide a variable range of tensions and effective diameters of support over a given width W or i.e., supported length of an organ.

FIG. 28a shows a sling 268 designed in the form of a balloon. At least one gas or media injector valve is required, and two gas/air injection valves 270 and 272 are shown, with one on each of ends 274 and 276. The function of the balloon construction is to provide an alternative apparatus and method of adjusting the tension and contact area on an organ supported by the sling. The sling 268 has angled laterally projecting spikes/protrusions 278 extending from the sling body 280 and serve the same purpose as the saw tooth protrusions of FIGS. 20c and 21a. The interior of the balloon sling 268 is more clearly indicated by the cross sectional view of FIG. 28b, with section B-B showing the relatively unexpanded cavity 282 formed by material 280. Air or other media injection will expand the cavity 282. As with the sling of FIG. 21a, sling 268 also has guide holes 284 and 286, with one hole on each end of the sling. FIG. 28c is another cross section B-B of the sling of FIG. 28a, except that it shows an additional material layer 288 applied over the material 280. As shown, layer 288 can be corrugated in shape to provide resistance between the sling and the organ. Material 288 can also be biodegradable material and can include the treatment substances as discussed above in reference to FIGS. 25 and 26.

FIGS. 29a-c illustrate an adjustable sling 290. The sling 290 is constructed in a similar manner to the sling of FIGS. 28a and 28b with a passage/lumen 292 extending from a first end 294 to a second end 296. The sling 290 differs from the sling of FIGS. 28a and 28b in that a flexible, elongated member 298 is installed through the passage/lumen 292. For descriptive purposes, the member 298 will be referred to as a “string” in the following specification and claims, but it is to be understood that the member 298 can be constructed from any of various materials and does not have to be a loop as illustrated. For example, the “string” could be a plastic ribbon, etc. The first length portion 300 of the sling and third length portion 302 include protrusions 304, similar to the protrusions 278 of FIG. 28a. The portions 300 and 302 in the embodiment of FIGS. 29a-c are constructed of flexible material that will collapse in folds upon application of pressure tangential to the length of the sling. The center portion 306 is similar to that shown in reference to FIG. 28a, configured to give support to an organ such as organ 308 of FIG. 29a. FIG. 29a shows the body organ 290 for purposes of illustration assumed to have dropped out of its normal position relative muscle tissue 310. Openings 312 and 314 are made through the muscle tissue 310 and the sling 290 is inserted through one of the holes, for example hole 314 under the organ 308 and out the other hole 312. The protrusions 304 serve to anchor the sling to the muscle tissue at the openings 312 and 314. Once the sling is in position, the protruding portions 316 and 318 of the first and second lengths 300 and 302 can be cut off flush with the surface of the tissue 310 as shown in FIG. 29b.

If the organ 308 needs more support, the sling 290 can be shortened by pulling the ends 320 and 322 of the “string” 298. If necessary, the ends of the collapsible first and third portions 300 and 302 can be supported by application of axial pressure on the portions 300 and 302 at the openings 312 and 314 so as to transfer the pressure caused by pulling the ends 320 and 322 of the string 298 to the sling, to cause the portions 300 and 302 to collapse as shown in FIG. 29b, resulting in a shorter sling 298, further resulting in the organ 308 being moved closer to the muscle tissue 310.

With the organ 308 in position subsequent to shortening the sling 290, the string 298 can be secured relative to the sling 290 so as to hold the sling in its shortened state. This can be done in a variety of ways that will be apparent to those skilled in the art. FIG. 29c shows the string 298 secured relative to the sling 290 by the tying of knots 324 and 326.

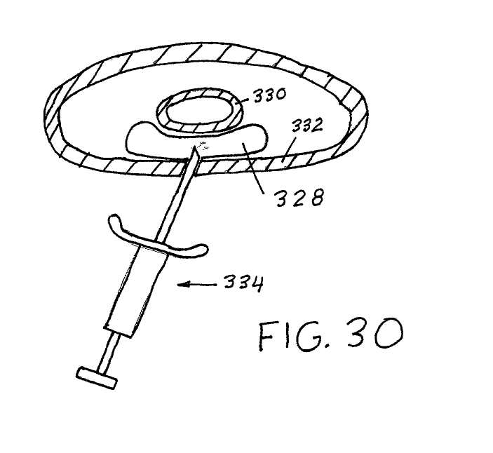

A further embodiment of the support structure of the present invention is illustrated as a method in FIG. 30. According to the method, liquid, gel or semi-solid 328 is injected between an organ 330 in need of support and an adjacent body part 332. An injector apparatus 334 can be of any type suitable to the particular substance being dispensed. The injected substance can be either self curing or can be a type that requires an induced environment such as a particular temperature, or applied electric or magnetic field or electrical current.

Although the present invention has been described above in terms of specific embodiments, it is anticipated that alterations and modifications thereof will no doubt become apparent to those skilled in the art. It is therefore intended that the following claims be interpreted as covering all such alterations and modifications as fall within the true spirit and scope of the invention.

It is claimed that:

1. An apparatus for supporting/suspending a body organ comprising:
(a) an elongated member for supporting a body organ constructed of material selected from a first group consisting of polymers and co-polymers, said member including
(i) a first length having a plurality of protrusions and a first end portion with a guide hole for attachment of a tool; and
(ii) a second length having a first end attached to a second end of said first length.

2. An apparatus as recited in claim 1 wherein said second length has a plurality of holes therethru for passage of a portion of said first length for forming an adjustable self locking band shaped structure for said supporting of a body organ.

3. An apparatus as recited in claim 1 wherein said elongated member further includes a third length having a first end attached to a second end of said second length forming a sling, and wherein said third length includes protrusions.

4. An apparatus as recited in claim 1 wherein said material further includes a treatment substance.

5. An apparatus as recited in claim 1 wherein a selected said material is biodegradable.

6. An apparatus as recited in claim 3 wherein a second end of said third length has at least one guide hole.

7. An apparatus as recited in claim 1 wherein said elongated member includes
(a) a first base layer constructed of said material; and
(b) a second layer formed on said first layer, said second layer constructed from material selected from said first group and from a second group consisting of a treatment substance.

8. An apparatus as recited in claim 1 wherein said member is constructed by a method of material interleaving.

9. An apparatus as recited in claim 8 wherein said member is textured to enhance tissue adherence.

10. An apparatus as recited in claim 1 wherein said member is constructed including one or more pockets containing a substance for providing shock absorption.

11. An apparatus as recited in claim 1 wherein said member includes a cavity construction for injection of a substance for providing adjustable tension or compressive force.

12. An apparatus as recited in claim 1 wherein said material is selected from a third group consisting of an FDA approved color dye and a color polymer blend to improve visibility against body tissue.

13. An apparatus for installing a structure for supporting a body organ comprising:
(a) an elongated, curved needle with a hooked tip at each of first and second ends of said needle; and
(b) a removable handle for attachment on a selected one of said first and second ends of said needle.

14. An apparatus as recited in claim 5 wherein said material is selected to achieve a desired mechanical property and absorption time in a body.

15. An apparatus as recited in claim 1 wherein said material changes length and shape in response to a change of temperature.

16. An apparatus as recited in claim 1 wherein said material changes length in response to application of electrical energy.

17. An apparatus as recited in claim 1 wherein said material changes in length in response to application of magnetic energy.

18. A method of supporting a body organ including placing an elongated support apparatus in contact with an exterior of said organ.

19. A method as recited in claim 18 wherein said support is a band placed around said organ.

20. A method as recited in claim 18 wherein said support is a sling having ends anchored in muscle tissue.

21. A method as recited in claim 18 wherein said support is a sling with an integrated band.

22. A method as recited in claim 18 wherein said support is constructed from material including a polymer.

23. A method as recited in claim 22 wherein said support is constructed from material selected from the group consisting of bioabsorbable and nonbioabsorbable polymers, pharmaceutical drugs and chemical agents.

24. A method as recited in claim 19 wherein said band is constructed with a self locking adjustable feature.

25. A method as recited in claim 20 wherein said sling is adjustable and self locking in length.

26. A method as recited in claim 18 wherein said support includes a cavity and a valve for injection of a substance for expanding said cavity for providing compression around said body organ.

27. A method as recited in claim 18 wherein said support is installed through an incision into a body cavity containing said organ.

28. A method as recited in claim 19 wherein said band is for treatment of sphincter deficiency.

29. A method as recited in claim 28 wherein said organ is selected from the group consisting of a urethra, a colon, a stomach, rectum and uterus.

30. A method as recited in claim 20 wherein said sling has a multi-layered construction with inner and outer extension lengths that can be pulled and pushed to adjust tension on an organ.

31. A method as recited in claim 18 wherein a length of said support is adjustable using thermal energy.

32. A method as recited in claim 18 wherein a length of said support is adjustable using electrical energy.

33. A method as recited in claim 18 wherein a length of said support is adjustable using magnetic energy.

34. A method as recited in claim 18 wherein said support is constructed of bioabsorbable polymers and copolymers selected to meet a desired mechanical property and a desired absorption rate.

35. An apparatus as recited in claim 3 wherein said protrusions provide adjustable self locking of said first and third lengths in body tissue.

36. A method of supporting a body organ comprising injecting a substance between said organ and another body part.

37. A method as recited in claim 36 further comprising curing said substance to a solid phase.

38. A method as recited in claim 37 wherein said curing is a method selected from the group consisting of application of heat, cold, radio frequency radiation, a magnetic field, and application of a chemical.
